(19) United States
(12) Patent Application Publication
(10) Pub. No.: US 2017/0072097 A1
(43) Pub. Date: Mar. 16, 2017

(54) INJECTABLE CHITOSAN SPONGES FOR ENHANCING BONE REGENERATION

(71) Applicant: THE ROYAL INSTITUTION FOR THE ADVANCEMENT OF LEARNING / MCGILL UNIVERSITY, Montreal (CA)

(72) Inventors: Lamees NAYEF, Montreal (CA); Mina MEKHAIL, Montreal (CA); Maryam TABRIZIAN, Longueuil (CA)

(73) Assignee: THE ROYAL INSTITUTION FOR THE ADVANCEMENT OF LEARNING / MCGILL UNIVERSITY, Montreal (CA)

(21) Appl. No.: 15/259,236

(22) Filed: Sep. 8, 2016

Related U.S. Application Data
(60) Provisional application No. 62/216,396, filed on Sep. 10, 2015.

(51) Int. Cl. A61L 27/20; A61L 27/54; A61L 27/56; A61L 27/50

(52) U.S. Cl. CPC A61L 27/20; A61L 27/50; A61L 27/54; A61L 27/56; A61L 2430/02; A61L 2400/06; A61L 2300/414; A61L 2300/45; A61L 2300/254

(57) ABSTRACT

It is provided a chitosan sponge for bone regeneration in a subject, comprising chitosan; a purine compound such as guanosine 5′-diphosphate (GDP); and at least one of a growth factor and a pyrophosphatase. Preferably the growth factor is BMP-7. The sponge is formed when the chitosan and the growth factor and/or pyrophosphatase is mixed with the purine compound such as guanosine 5′-diphosphate.

### TECHNICAL FIELD

It is provided a chitosan sponge for bone regeneration.

### BACKGROUND

Small bone fractures can heal by themselves without need for surgery;

however, if the fracture is large, an intervention is necessary. The healing process post-intervention is made even more complicated in elderly patients who suffer from diabetes, deficiencies in blood supply or infection. Patients suffer large bone losses due to accidents, infections or removal of cancerous tumours. Critical size bone defects (CSBD) are non-healing injuries involving the loss of large segments of bone that cannot be regenerated spontaneously by the body, and therefore require a therapeutic intervention. Grafting autologous bone is the most commonly used method to stimulate and accelerate bone growth in such cases. However, invasive surgeries are needed to both harvest and implant these grafts, which increases risk of post-surgical infection and prolong hospitalization time. Therefore, there remains a need to design viable therapeutic alternatives to enhance bone regeneration. Injectable scaffolds are an emerging class of biomaterials that solidify into three-dimensional (3D) substrates after application in vivo, thus eliminating the need for invasive surgery to implant the scaffold. In addition to the favorable hydrated environment they provide for cells, the gelation mechanism of these scaffolds allows easy encapsulation of other mineralization stimulants like stem cells, bioceramics and growth factors.

It would be highly desirable to be provided with a scaffold composition for enhancing bone regeneration.

### SUMMARY

One aim of the present description is to provide a gelling composition comprising chitosan; a purine compound; and at least one of a growth factor and a pyrophosphatase.

In an embodiment, the purine compound is at least one of a guanosine 5′-diphosphate (GDP), adenosine triphosphate (ATP), adenosine diphosphate (ADP), guanosine triphosphate (GTP), a substituted purine and a tautomer thereof.

In another embodiment, the purine compound is guanosine 5′-diphosphate (GDP).

In a further embodiment, the composition comprises the growth factor and the pyrophosphatase.

In an embodiment, the growth factor is at least one of a platelet-derived growth factor (PDGF), an insulin-like growth factor (IGF), a fibroblast growth factor (FGF), a transforming growth factor (TGF), an epidermal growth factor (EGF), a nerve growth factors (NGF), a vascular endothelial growth factor (VEGFs), and a bone morphogenetic protein (BMP).

In another embodiment, the growth factor is at least one of PDGF, BMP-7, BMP-2, TGF-β, IGF-I, IGF-II, and bFGF.

In a further embodiment, the composition described herein comprises 0.1 to 10 μg of the growth factor.

In an embodiment, the composition described herein comprises 1 μg of the growth factor.

In a supplemental embodiment, the composition encompassed herein is being formulated for an injection.

In a other embodiment, the composition is formulated for an injection by a Twin-Syringe Biomaterial Delivery System.

In an embodiment, the composition described herein is for bone regeneration.

In an embodiment, the composition described herein is for bone regeneration in a subject with a fracture or a critical size bone defect (CSBD).

In another embodiment, the subject is an animal or a human.

In an embodiment, the composition described herein is for the sustained release of the growth factor in a subject.

In an embodiment, it is provided a method of encapsulating a pyrophosphatase in a sponge comprising the steps of mixing a chitosan solution with a pyrophosphatase containing solution; adding a solution of a purine compound; and mixing the solution of the purine compound with the chitosan solution and pyrophosphatase containing solution forming the sponge.

In another embodiment, it is also provided a method of encapsulating a growth factor in a sponge comprising the steps of mixing a chitosan solution with a growth factor containing solution; adding a solution of a purine compound; and mixing the solution of the purine compound with the chitosan solution and growth factor containing solution forming the sponge.

In an embodiment, it is further provided a kit comprising a chitosan solution; a solution containing at least one of a growth factor and a pyrophosphatase; and a purine compound solution; wherein a gel is formed when the chitosan solution and the solution containing at least one of the growth factor and the pyrophosphatase are mixed with the purine compound solution.

In an embodiment, the chitosan solution, the solution containing at least one of the growth factor and the pyrophosphatase and the purine compound solution are manufactured in separate syringes.

In another embodiment, the chitosan solution and the solution containing at least one of the growth factor and the pyrophosphatase are manufactured in a double-barrel syringe.

In an embodiment, it is further provided a method for stimulating bone regeneration in a subject comprising administering the composition described herein to the subject.

In another embodiment, it is provided the use of the composition described herein for stimulating bone regeneration in a subject.

In another embodiment, it is provided the use of the composition described herein in the manufacture of a medicament for stimulating bone regeneration in a subject.

In an embodiment, the subject has a fracture or a critical size bone defect (CSBD).

### BRIEF DESCRIPTION OF THE DRAWINGS

Reference will now be made to the accompanying drawings.

FIG. 1 illustrates an in vivo assessment of sponge injectability and localization showing that a chitosan sponge according to an embodiment takes the full size and shape of a CSBD after injection in a rat model, wherein in (A) the femur of a rat model is exposed after removal of surrounding skin and muscle; (B) the fixator is attached to the femur and an osteotomy of a 6 mm bone segment is performed to create the critical size defect; (C) the excised bone segment; (D) the soft tissue and skin are sutured and the chitosan/GDP is injected into the critical size defect; and (E) the skin and muscle are removed to observe the chitosan sponge formation.

FIG. 2 illustrates an in vitro assessment of a sponge according to one embodiment morphology, encapsulation and release kinetics, wherein showing in (A) a low magnification SEM of the chitosan sponge demonstrating the overall microstructure; (B) a higher magnification SEM of the chitosan sponge demonstrating the microenvironment which is highly porous with interconnected pores, and is composed of fused nanoparticulates; (C) TEM of individual nanoparticulate structures that form the chitosan sponge; (D) the encapsulation efficiency of the chitosan sponge as compared to that of liposomes; (E) the cumulative release of BMP-7 from the chitosan sponge over a 30 day period together with an image of the chitosan sponges at day 0 and day 30 demonstrating sponge degradation; and in (F) the fitting the % cumulative release data (up to 60% release) into the Korsmeyer-Peppas release kinetics model, the data fitting with an R2 value of 0.98 and a linear equation y=0.67×+0.96.

FIG. 3 illustrates the ALP activity of MC-3T3 cells in response to bioactive BMP-7 released from chitosan sponges according to one embodiment, showing normalized ALP activity of MC-3T3 cells after exposure to sponges containing 1 μg BMP-7, blank sponges and no sponges (control) after 1, 3 and 6 days, all results are presented as mean ±SEM (within each time point: *P<0.05, **P<0.01, ***P<0.001, within each experimental group: #P<0.05, ##P<0.01, ###P<0.001).

FIG. 4 illustrates a pyrophosphate luminescence assay results showing the levels of pyrophosphate in media after one week of incubating sponges containing 0.1,1 and 10 Units of PPtase as well as blank sponges and controls without sponges or PPtase (***P<0.001).

FIG. 5 illustrates the effect of chitosan sponges according to an embodiment on mineralization as assessed using alizarin red staining in an indirect cell culturing method, showing sponge with PPtase (A), blank sponge (B), BMP-7 and PPtase administered directly into the media (C), BMP-7 in media (D), PPtase in media (E), and control with no treatment (F); each panel has a representative picture of the well before alizarin red staining, the same well after staining with alizarin red, and a representative light microscope image of the cell monolayer prior to alizarin red staining; and in (G) quantification of the alizarin red dye in each experimental group (*** P<0.001).

FIG. 6 illustrates chitosan sponge biocompatibility tests using heamatoxyline and eosin staining. Chitosan sponges where subcutaneously injected in a rat before euthanasia at 15, 30 and 60 days post injections. Skin samples were collected and fixed in 4% buffered formaldehyde and processed for H and E staining. The chitosan sponges are colored (black arrows). By Day 30 the chitosan sponges are being degraded and engulfed by the immune cells. The chitosan sponges are almost completely degraded by day 60. Insets: Wright's staining. Neutrophils and macrophages are surrounding the chitosan sponges-pinkish red (day 30). Their numbers were significantly reduced by day 60 in parallel with the biodegradation of the chitosan.

FIG. 7 illustrates microCT imaging on CSD rats right after the surgery and beyond 13 weeks. Black arrow: ectopic bone formation in the surrounding tissues of the rats that received the Medtronic INFUSE.

### DETAILED DESCRIPTION OF THE PREFERRED EMBODIMENT

In accordance with the present description, there is provided a gelling composition comprising chitosan; a purine compound; and at least one of a growth factor and pyrophosphatase.

Accordingly, the gelling composition comprises a purine compound consisting of a heterocyclic aromatic organic compound. It consists of a pyrimidine ring fused to an imidazole ring. Encompassed herein are purines known in the art, which include substituted purines and their tautomers. For example, purines encompassed herein are guanosine 5′-diphosphate (GDP); adenosine triphosphate (ATP), adenosine diphosphate (ADP) and guanosine triphosphate (GTP).

Regeneration of large amounts of bone becomes necessary for patients with critical size bone defects (CSBD) or fracture for example, in order for them to return to normal life. The composition described herein essentially comprises a hydrogel scaffold that can be used as a drug delivery device to speed up bone tissue growth. The composition is highly desirable since it is injectable, which allows for an administration minimally invasive, and it can fill bone defects regardless of the irregular geometry of the defects. Moreover, controlling hydrogel degradation and swelling can provide a sustained release of therapeutic agents. The most commonly delivered therapeutics to accelerate bone growth includes bone morphogenetic proteins, especially BMP-7 and BMP-2. Alternatively, growth factors including platelet derived growth factor (PDGF), fibroblast growth factors (FGF), transforming growth factor beta (TGF-β) and insulin growth factor (IGF) have also been used to stimulate bone growth. Growth factors that encourage bone formation through encouraging angiogenesis like vascular endothelial growth factors (VEGFs) have also been used.

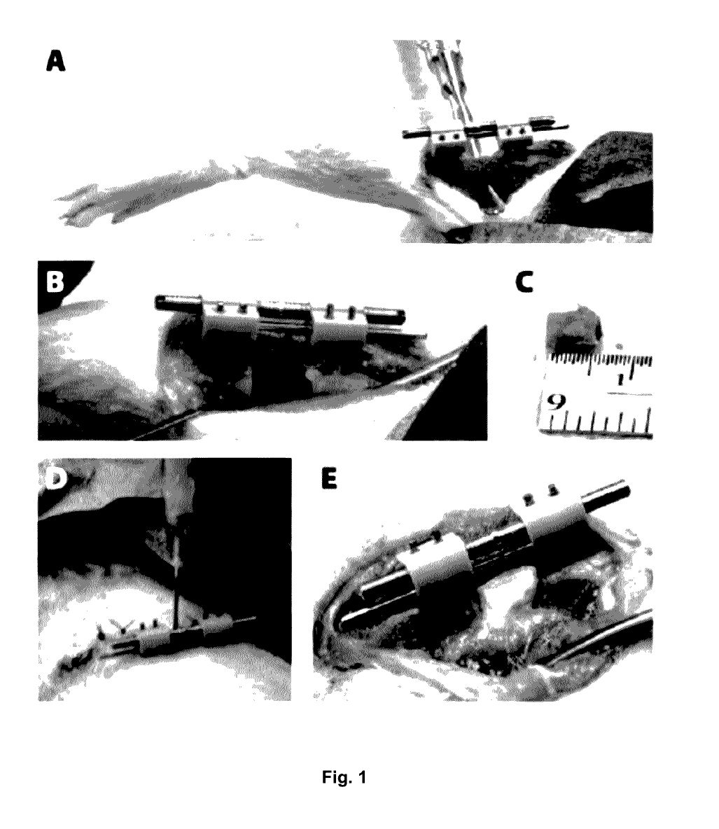

It is thus disclosed herein a rapidly-gelling injectable chitosan sponge for bone regeneration in CSBD applications. The sponge is desirable notably because when injected it takes the full size and shape of the CSBD, allowing evenly distributed regeneration over all areas of the defect (see FIG. 1E). The high encapsulation efficiency of BMP-7 (84.3 ±2.3%) within the sponges and the controlled sustained release over 30 days are disclosed. Moreover, the bioactivity of the released BMP-7 was confirmed using an ALP assay. PPtase encapsulated in the sponges was shown to reduce PPi levels to near zero values, and was shown to significantly enhance biomineralization. In fact, the sponges encapsulating PPtase performed similarly in response to the direct addition of BMP-7 into the media. The use of low concentrations of BMP-7 in the sponge act as a chemoattractant of MSCs, and the use of encapsulated PPtase improves mineralization.

The gelation of the chitosan sponge described herein occurs readily upon mixing chitosan and guanosine 5′-diphosphate (GDP) solutions due to ionic interactions between the anionic phosphate groups in GDP and the cationic amine groups in chitosan, as described in WO 2014/036649, the content of which is incorporated herein in its entirety. It is disclosed herein the use of rapidly forming three-dimensional (3D) chitosan sponges in situ upon mixing of two injectable solutions using GDP (Guanosine 5′-Diphosphate) as an anionic crosslinker for chitosan. The ionic attractions between the phosphate and amine groups occur very rapidly upon mixing and form an intact chitosan sponge at a 5 to 6 pH range. The sponge described herein also occurs when chitosan is mixed with adenosine diphosphate (ADP) as tested.

The sponge described herein is one of the most rapidly-gelling system currently available (tgel<1.6 sec). Fast gelation allows for the efficient entrapment of growth factors in vivo, in addition to excellent localization post-injection (Mekhail et al., 2013, Adv Healthc Mater, 2: 1126-1130). However, it has been established herein that degradation products of the chitosan sponges as described in WO 2014/036649 contain pyrophosphate (PPi), a known inhibitor of mineralization. It has been well established that increasing the ratio of pyrophosphate to phosphate ions (PPi/Pi) significantly reduces mineralization. PPi is produced by enzymatic cleavage of GDP into guanosine and PPi by alkaline phosphatase (ALP).

It is provided a gelling composition comprising chitosan and guanosine 5′-diphosphate (GDP), wherein the composition forms a gel when the chitosan is mixed with the GDP at a pH range from 5 to 6.

The disclosed gel or sponge could be injected with known techniques and devices, such as the Twin-Syringe Biomaterial Delivery System (M-System™).

Chitosan is an amino polysaccharide obtained by partial to substantial alkaline N-deacetylation of chitin also named poly(N-acetyl-D-glucosamine), which is a naturally occurring biopolymer found in exoskeleton of crustaceans, such as shrimp, crab and lobster shells. Chitosan contains free amine (-NH₂) groups and may be characterized by the proportion of N-acetyl-D-glucosamine units and D-glucosamine units, which is expressed as the degree of deacetylation (DDA) of the fully acetylated chitin polymer. The properties of chitosan, such as the solubility and the viscosity, are influenced by the degree of deacetylation (DDA), which represents the percentage of deacetylated monomers, and the molecular weight (Mw).

Accordingly, the present disclosure provides an approach to overcoming this inhibitory effect and still enhances biomineralization using the chitosan sponges.

Overall, the injectable chitosan sponge disclosed herein provides both a scaffolding material, which is highly porous, to promote osteoblast infiltration and differentiation, as well as a mechanism to release phosphate ions into the milieu to accelerate mineralization and bone regeneration .

The composition described herein encapsulates bone morphogenetic protein 7 (BMP-7), an osteogenic factor that is one of the most powerful in inducing mineralization, in the chitosan sponges. BMP-7 can counteract the effects of PPi and can also act as a chemotactic agent to attract more mesenchymal stem cells into the scaffold during bone regeneration in vivo. The sponge's rapid gelation ensures high encapsulation efficiency of BMP-7 in vivo, and decreases unwanted diffusion of BMP-7 to surrounding tissues. Moreover, BMP-7 is expensive, and therefore a controlled release system can reduce the concentration required to induce mineralization.

The composition described herein further encapsulates the enzyme pyrophosphatase (PPtase) in order to reverse the inhibitory effect of PPi. PPtase delivered gradually from the sponge cleaves each PPi molecule formed from GDP into 2 Phosphate ions (Pi), which significantly increases the Pi/PPi ratio and thus improves mineralization.

PPtase is an enzyme that is encapsulated in the sponge disclosed herewith in order to eradicate the inhibition of mineralization caused by the increase in the ratio of PPi/Pi discussed herein and converting it to a source of phosphate ions which are known to enhance biomineralization especially when exposed to the cells in small amounts for prolonged times. The phosphate ions both act as building blocks for the mineral, hydroxyapatite and as signaling molecules that upregulate mineralization. Contrary to known scaffolds that work by gradually supplying phosphate ions to enhance bone regeneration and that have always been ceramic or had a ceramic component, the sponge described herein represent a soft scaffold with no ceramic components, which is able to enhance biomineralization by the gradual delivery of phosphates through the use of its protein delivery property to enzymatically convert the sponge's biodegradation products to phosphate ions.

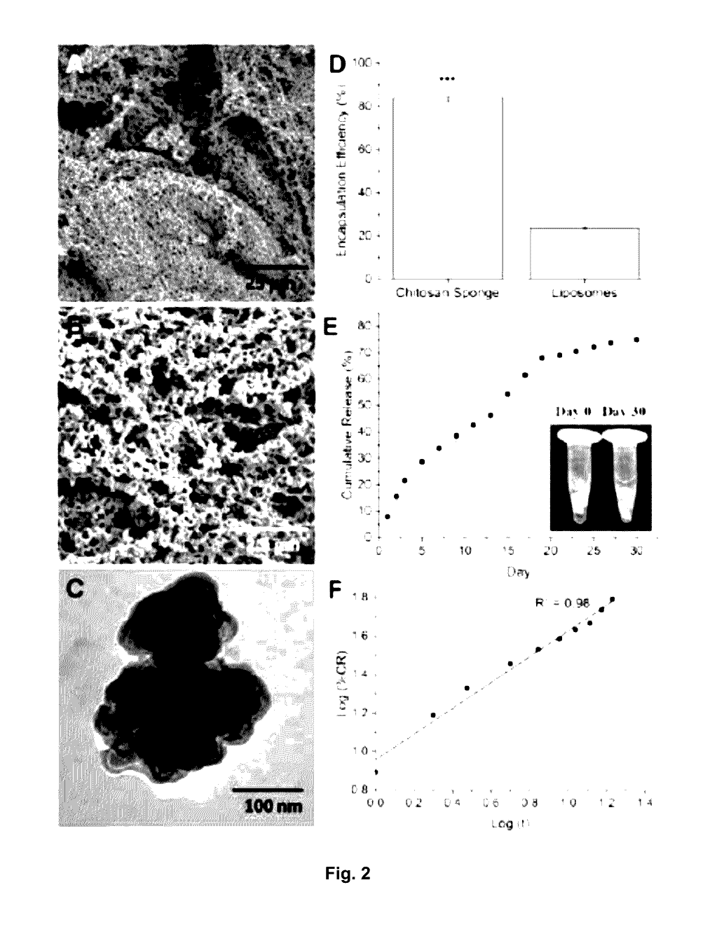

In an embodiment described herein, the chitosan sponge disclosed herein is a rapid gelling system (tgel<1.6 seconds) due to ionic interactions between the anionic phosphate groups in GDP and the cationic amine groups in chitosan. Scanning electron microscopy (SEM) images of the chitosan sponge revealed its highly porous nature and the excellent interconnectivity between the pores (see FIGS. 2A and B). This microenvironment makes the chitosan sponge a suitable candidate for encapsulation and release of proteins. Furthermore, the nanoporous microenvironment of the chitosan sponge provides a tortuous path that hinders the burst release of growth factors such as BMP-7 and instead, allows extended controlled release. Transmission electron microscopy (TEM) images of GDP-crosslinked chitosan (at very low concentrations) revealed the building components of the chitosan sponges described herein, which are nanoparticulate structures of 140 nm in size (FIG. 2C). At high concentrations these nanoparticulates aggregate together to form the chitosan sponge. The hydrophilicity of these nanoparticulates makes the chitosan sponge encompassed herein a suitable carrier for encapsulating proteins and delivering them via diffusion. Moreover, since the sponge structure is stabilized via ionic crosslinking, as opposed to the stronger covalent crosslinking, protein release in the short term would occur due to hydrolytic and enzymatic degradation of both chitosan and GDP.

As encompassed herein, the composition disclosed comprises the scaffold described herein as a means to deliver growth factors in order to stimulate bone regeneration. Growth factors are polypeptides which interact with specific cell surface receptors. Examples of growth factors encompassed herein, but not limited to, are growth factors selected from platelet-derived growth factors (PDGFs), insulin-like growth factors (IGFs), fibroblast growth factors (FGFs), epidermal growth factors (EGFs), nerve growth factors (NGFs), transforming growth factors (TGFs), encouraging angiogenesis like vascular endothelial growth factors (VEGFs), and bone morphogenetic proteins (BMPs). More particularly, the growth factor encapsulated in the scaffold described herein can be PDGF, BMP-7, BMP-2, TGF-β, IGF-I, IGF-II, and/or bFGF.

BMPs, which are part of the TGF-9 superfamily, are characterized by their unique ability to induce osteoblastic differentiation. BMP-7 is a growth factor that has attracted much attention due to its powerful osteogenic activity. However, direct injection of BMP-7 is undesirable since it has a short half-life in vivo (30 minutes) and can readily diffuse to surrounding tissue. Increasing the amount of BMP-7 injected allows better bone regeneration, but has also been connected with ectopic bone growth, increased side effects on surrounding organs, increased risks of cancer and sometimes the stimulation of feedback control mechanisms that cause bone resorption. The chitosan sponge disclosed herein provides a mean for the encapsulation and controlled release of BMP-7. BMP-7 was entrapped within the sponge with an 84.3±2.3% efficiency as compared to 23.8±0.46% in liposomes, which are widely used for drug delivery applications (FIG. 2D). This comparison demonstrated the superiority of the chitosan sponge for BMP-7 entrapment. There was no burst release observed with the chitosan sponges, with only 7% of the BMP-7 released at day 1, and 50% by day 15 (FIG. 2E); this sustained release was provided for more than 30 days.

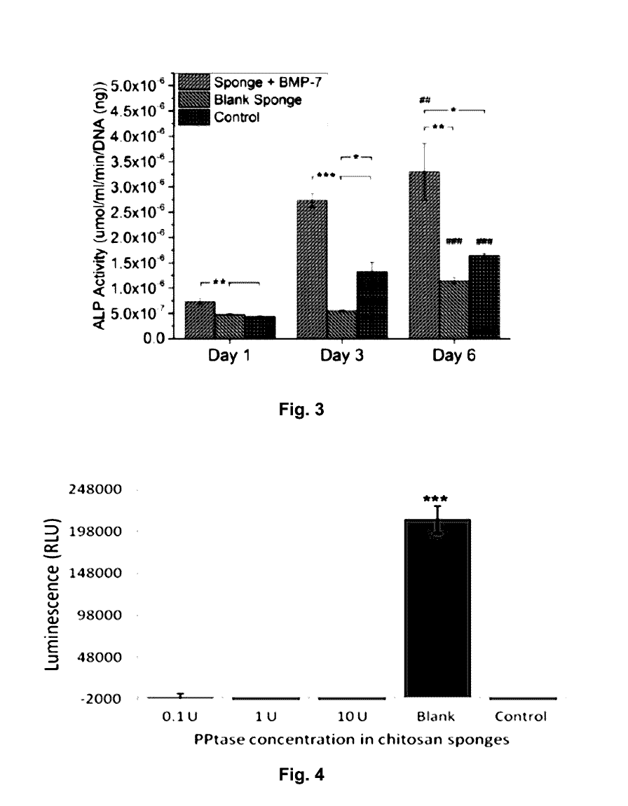

Such a controlled release is highly beneficial in a clinical setting, especially since the currently marketed bovine collagen sponges' result in loss of 30% of loaded BMP instantly after implantation, which limits the ability to achieve prolonged delivery. Moreover, providing a controlled release of BMP-7 for a prolonged period of time (>30 days) can lead to the formation of high quality bone. The data from the release kinetics study best fit (R2=0.98) the Korsemeyer-Peppas release kinetics model (FIG. 2F). From the slope and intercept of the fitted data ‘Km’ and ‘n’ are 0.96 and 0.67 respectively. According to the Korsemeyer-Peppas model a ‘n’ value of 0.67 shows that release of BMP-7 from the sponge is occurring due to diffusion and degradation. This was confirmed by observing the sponge at day 0 and day 30, where the chitosan turns yellowish in colour at day 30, resembling chitosan degradation (FIG. 2E). The bioactivity of released BMP-7 was also confirmed by measuring changes in ALP activity of mouse pre-osteoblast cells (MC3T3) exposed to sponges containing 1 μg of BMP-7 (see FIGS. 3A and B).

ALP is a membrane bound enzyme that increases bone mineralization. ALP activity is known to rise as osteogenesis progresses during the early stage of differentiation and then decreases at later osteogenic stages. The ALP activity of MC-3T3 cells was normalized to total DNA to ensure that increases in ALP activity were due to increases in osteogenic activity rather than increases in cell number. Results showed that at all time points, the normalized ALP activity of cells supplied with BMP-7 from loaded sponges was greater than cells from both control groups (FIG. 3). ALP activity of MC-3T3 cells in all three test groups increased with time, indicating increasing osteogenic activity as is expected with osteoblastic cells. ALP activity of cells exposed to BMP-7 from chitosan sponges did not increase significantly after the third day probably due to the progression to more advanced stages of bone growth, which is associated with reduced ALP activity.

The encapsulation of PPtase in the chitosan sponge as described herein to cleave PPi into Pi provided to enzymatically convert the sponge's biodegradation products to phosphate ions. Once the sponge is placed in media, GDP leaches out from the sponge and is enzymatically broken down by ALP (produced by differentiating MC-3T3s) into guanosine and PPi. Therefore, incorporation of PPtase in the sponges enzymatically cleave the PPi into 2 Pi, which increases the concentration of Pi (a building block of hydroxyapatite and regulator of many genes controlling mineralization), and in turn enhances biomineralization.

A PPi assay was used to measure the availability of PPi in the media containing sponges with different PPtase concentrations, as well as blank sponges and the control that did not have a sponge or PPtase. The results confirmed that the blank sponges released large quantities of PPi, and that the addition of PPtase, as low as 0.1 Units/sponge, can cleave all the PPi in the media (FIG. 4).

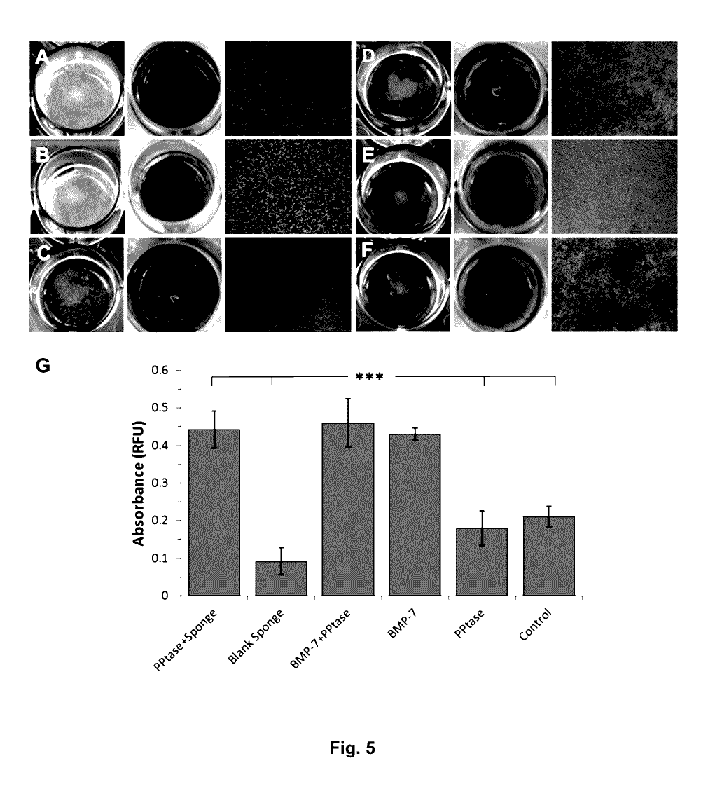

It is demonstrated herein that the sponges containing PPtase significantly (P<0.001) enhanced mineralization as compared to blank sponges (4 fold increase), sponges containing 1 μg of BMP-7 and the sponges which had the combination of BMP-7 and PPtase. Moreover, it is demonstrated that the sponges +PPtase had the same effect on mineralization as direct application of 1 μg of BMP-7 (FIGS. 5 A, D and G). This experiment also confirmed that the PPtase can only have an enhanced effect on mineralization when there is a source of PPi to be cleaved into Pi. When there was no PPi (i.e. just media with no sponge), PPtase did not further enhance mineralization as compared to the control (FIGS. 5E and F). Moreover, PPtase did not cause any further enhancement of mineralization when administered with BMP-7 directly into the media (FIGS. 5C and E). Even though the blank sponges showed a reduction of mineralization (FIG. 5B) as compared to the controls (due to the release of PPi), it was very interesting to observe that all the MC-3T3 monolayers exposed to the sponge were more opaque compared to the all other controls without sponge. It can also be observed clearly from the controls, that the more opaque areas correspond to mineralization (FIGS. 5 C, D, E and F). Since mineralization has two components, namely the organic osteoid matrix, and inorganic hydroxyapatite, one can conclude that colocalization of the opaque region and the alizarin stain corresponds to mineralized osteoid, while the opaque regions that do not contain alizarin stain are unmineralized osteoid. Light microscopy images of the opaque regions revealed dark nodules on top of the cell monolayer that are responsible for the scattering of light in the photographic image. The growth of such nodules, also called biomineralization foci (BMF) and the progression of their mineralization from organic osteoid to mineralized osteoid have been described in literature. The addition of Pi has also been reported to participate in driving the initiation of mineralization of those nodules. The nodules were over-expressed on the cell monolayer when exposed to all the sponge groups as compared to all other controls. However, only a portion of this opaque region was stained with alizarin red. This result demonstrates that the chitosan released from the sponges (due to degradation) promotes osteoid deposition, but not mineralization.

The incorporation of PPtase can thus lead to both an enhancement in osteoid production and mineralization. Chitosan has been previously shown to improve collagen (the main component of the organic osteoid matrix) deposition by MC-3T3 cells.

Bone regeneration was assess in vivo in rats that were sacrificed several days to weeks after surgery and the tissue processed for histological evaluation and micro computed tomography (pCT) analyses.

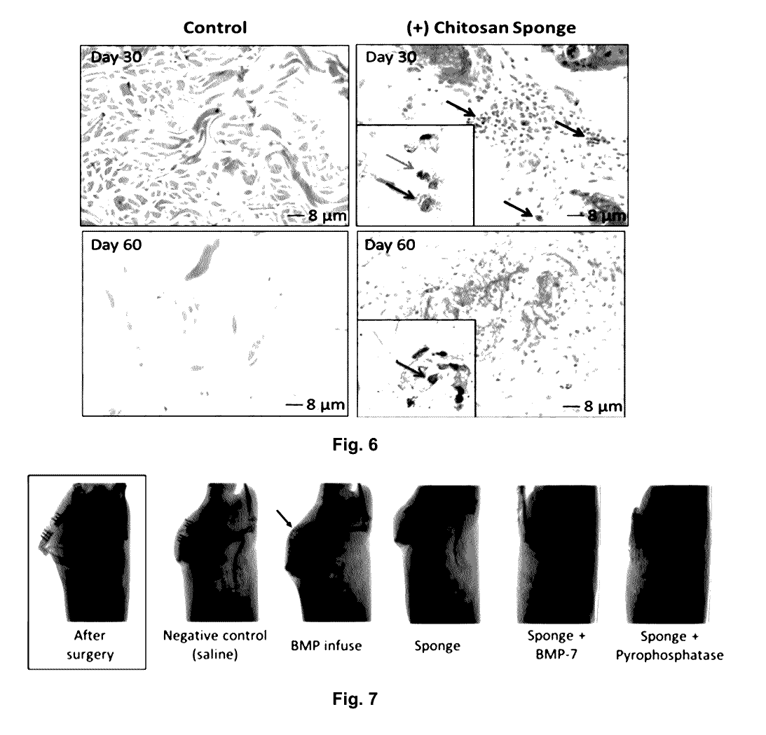

In order to first assess in vivo biocompatibility testing of the chitosan sponges in rat subcutaneous injection model, the sponges were prepared and brought to the surgical suite for injection. The rat skins were collected and tested 15, 30 and 60 days post injections. The results shows that chitosan sponges likely elicit a minimal immune reaction 15 days post injection (FIG. 6), but their biodegradation is evident by day 30, where macrophages are shown to engulf the chitosan sponges, and facilitates for their systemic clearance. The chitosan sponges are completely degraded by day 60. FIG. 6 shows the recruitments of neutrophils and macrophages (after staining with Wright's stain) on days 15 and 30 post injection, whereas by day 60 these cells are no longer detected. These results confirmed the biocompatibility and safety of the chitosan sponges described herein.

In order to investigate the extent of bone formation induced by the chitosan sponge loaded with therapeutics as drug delivery system to support and accelerate bone healing in a rat critical size defect model, twenty rats were separated into 5 different groups. Two negative control groups; normal saline or chitosan sponges where injected within the bone gaps of a CSD. Medtronic infuse bone grafts mixed with human recombinant BMP-2 was inserted in the CSD (positive control group). And two test groups; chitosan sponges blended with either BMP-7 or PPtase. The results successfully showed that the chitosan sponges containing PPtase supports the formation of calcified tissue in the bone defect. The Medtronic INFUSE used as a positive control on the other hand resulted in the formation of ectopic bone in the surrounding tissues, manifested clinically on the rat femur as a bulky swollen mass that hindered normal gait. Moreover, bone was not detected within the CSD in negative control groups or in the chitosan sponge+BMP-7 group (FIG. 7). The concentration used in this experiment were not optimal and is it expected that as encompassed herein, a chitosan sponged with BMP-7 will stimulate bone formation.

In an embodiment, it is provided a sponge encapsulating both PPtase and BMP-7, or sponges encapsulating one of PPtase and BMP-7 and supplementing the other separately will improve mineralization and promote MSC infiltration that makes such sponges useful for improving bone regeneration in CSBD.

Accordingly, the unique combination of the injectable chitosan sponge and Pptase provides a cheap and effective system that can be applied using minimally invasive surgery to increase mineralization and improve osteoblast differentiation, and improve bone regeneration in fractures. The chitosan sponge provided herein can be used to improve bone regeneration in these situations and alleviate the need for extracting autologous bone for grafting. Moreover, the sponge described herein is an injectable system that can encapsulate proteins (enzymes and growth factors) and anti-inflammatory agents to improve both osteogenesis and vascularisation. The chitosan sponge can gel rapidly, which guarantees localization at the site of injection, and also the ability to assume the irregular shape of the defect. The localized release of Pptase will also accelerate bone formation.

Known and commercially-available bone graft substitutes utilize the release of growth factors (such as rhBMPs) to induce bone regeneration. Two of the major products that have dominated the market in this field are Medtronic's INFUSE™ and Stryker's OP-1™; both are collagen-based materials that release rhBMP-2 and rhBMP-7 respectively. However, both systems use large doses of BMP (in the mg range) to induce the required osteogenic effect, due to the short-half-life of BMPs in vivo, which in turn makes these systems very expensive. Moreover, BMP diffusion away from the application site has been shown to lead to unwanted ectopic bone growth and various medical complications. On the contrary, the sponge disclosed herein uses pyrophosphatase, which is much cheaper compared to BMPs. Moreover, the sponge disclosed herein will not induce any ectopic bone growth that can be caused by using BMPs. The slow release of pyrophosphatase from the sponge can further reduce the pyrophosphate concentration in the bone milieu and improve bone formation. The osteogenic effect of the sponge described herein can be further enhanced by including small amounts of BMPs, which are closer to physiological conditions.

The present description will be more readily understood by referring to the following examples.

### EXAMPLE I

### Preparation of Chitosan Sponges

A chitosan solution is prepared as previously reported (Mekhail et al., 2013, Adv Healthc Mater, 2: 1126-1130). Briefly, 60 mg of chitosan is dissolved in 10 ml of 0.06M HCl solution under magnetic stirring for 30 minutes. The pH of the solution is adjusted to 6 using a 1M sodium bicarbonate solution. A GDP solution (100 mg/ml) is prepared by dissolving GDP in distilled water. The chitosan and GDP solutions are sterilized by filtration through 0.22 μm syringe filters under a laminar hood. To prepare sponge containing BMP-7, 1 μg of BMP-7 is dissolved in 100 μl of sterile distilled water and added to a sterile LoBind eppendorf tube containing 1.6 ml of chitosan solution then mixed thoroughly. Immediately after, 0.3 ml of the GDP solution is rapidly injected into the chitosan solution to form the sponge. The eppendorf is closed and inverted repeatedly to ensure complete gelation. The sponge is then removed using tweezers, placed in another LoBind tube and rinsed once with PBS. The supernatant left from the sponge formation is centrifuged for 1 minute to pellet down any sponge debris and the volume of supernatant is measured. ELISA is then used to determine the concentration of the free BMP-7 in the supernatant and the weight of free BMP-7 (Wfree) is determined. The entrapment efficiency (EE) is calculated using Equation (1).

$$EE ( \% ) = \frac{{W}_{initial} - {W}_{free}}{{W}_{initial}} \times 100 \qquad ( 1 )$$

For comparison, BMP-7 loaded liposomes are fabricated using a previously reported method (Haidar et al., 2009, J Biomed Mater Res A, 91: 919-928) and were separated from free BMP-7 using size exclusion chromatography. The encapsulation efficiency of the separated liposomes is calculated by their dissolution using 0.1% TritonX™ and quantifying the encapsulated BMP-7. A Hitachi S-4700 Field Emission Scanning Electron Microscope (FE-SEM) at 2 KeV and a current of 10 μA and a cryo-TEM (FEI Tecnai G2 Spirit) employed at 120 KeV are used to observe the micro-architecture and building blocks of the sponge.

### EXAMPLE II

### Protein Release and Activity of Chitosan Sponges

A 0.1% w/v BSA in PBS solution is used as the protein release buffer for preserving protein activity. The sponges are placed in 0.5 ml of the buffer and incubated at 37° C. For the first three days the release buffer is removed daily, stored at −20° C. and is replaced with a fresh batch of release buffer. For all the remaining time intervals, the release buffer is collected every other day for one month. ELISA is used to measure the BMP-7 concentration in the release buffer every week to avoid loss of BMP-7 activity due to prolonged storage conditions as previously observed, and the weight of released BMP-7 is calculated (Ws). It is important to note that a new standard curve is prepared with every ELISA measurement. The absolute release (At) at each time interval is measured using Equation (2). Cumulative release (CR) is then calculated by the summation of “At” over a period of 30 days as shown by Equation 3.

$$\begin{matrix}{A}_{t} ( \% ) = \frac{{W}_{s}}{{W}_{initial}} \times 100 & ( 2 ) \\ CR ( \% ) = {\sum }_{t = 1}^{30} {A}_{t} & ( 3 )\end{matrix}$$

### EXAMPLE III

### Chitosan Sponges Bioactivity Measured by an Indirect Cell Culturing Technique

MC-3T3 cells are expanded in α-MEM supplemented with 10% Fetal Bovine Serum and 1% Penicillin-Streptomycin. Cells are then trypsinized and cultured in 24 well plates at a density of 1.5×104 cells/well. Differentiation media is then prepared by supplementing α-MEM (containing FBS and PenStrep) with 2.16 mg/ml β-Glycerophosphate and 50 μg/ml ascorbic acid. Cells are cultured in differentiation media for at least 4 days before beginning any experiment in order to better assess the effect of BMP-7 and PPtase.

Sponges containing 1 μg BMP-7 and blank sponges (negative control) are placed in hanging cell inserts and fitted on top of the monolayer of MC-3T3 while making sure the sponge is fully immersed in media. Cells not exposed to chitosan sponges are also used as controls. At day 1, 3 and 6 the sponges are removed and the cells washed three times with sterile PBS. The standard protocols provided by the manufacturer are then used to graph a standard curve for each experimental and to quantify both ALP using the ALP assay, and DNA using picogreen assay.

For measuring pyrophosphate release, empty sponges are placed in hanging cell inserts and fitted on top of MC-3T3 monolayers as in the indirect cell culture method. The cell monolayers are allowed to grow for 10 days at 37° C. in the incubator. In the same plate control cell monolayers without any inserts are used as negative controls. Media in contact with the group of cells grown with the sponge and the control cell group is collected on the 10th day and a PPiLight™ inorganic pyrophosphate assay (Lonza, US) used to measure pyrophosphate (PP) levels in both cell groups.

The effect of pyrophosphatase on pyrophosphate availability is assessed by encapsulating 0.1, 1 and 10 U of PPtase in different chitosan sponges. Indirect cell culturing is used to assess the activity of pyrophosphatase released from these sponges and its efficiency in lowering PPi levels. MC-3T3 cell monolayers from different groups are allowed to grow for 10 days at 37° C. in the incubator. Media from all groups is collected in the 10th day and tested using PPiLight™ inorganic pyrophosphate assay to test the activity of released pyrophophatase.

### EXAMPLE IV

### In Vitro Mineralization of Chitosan Sponges

Six groups were investigated to assess the efficiency of the chitosan sponge in inducing mineralization by itself, and with PPtase. The mineralization caused by these groups was compared to mineralization of MC-3T3 cell monolayers stimulated by direct injection of the same proteins to the media. Group 1 consisted of MC-3T3 cell monolayers in wells with inserts holding sponges loaded with 1U pyrophosphatase. Group 2 consisted of cell monolayers with inserts holding an unloaded sponge and was used as a negative control. Groups 3 to 5 consisted of cell monolayers with media injected directly with the same components as group 1. Group 6 consisted of cell monolayers without any injections to their media and was kept as a negative control. MC-3T3 cell monolayers for all groups were allowed to grow in 24 well plates under differentiation media for 14 days at 37° C. prior to starting the experiment. Sponges were then added to groups 1 and 2 for indirect cell culture and injections made to groups 3-6. Cells from all groups were allowed to grow for another 14 days in differentiation media at 37° C., with media change performed once every 5 days. Mineralization in all groups was then evaluated and quantified through staining with alizarin red.

### EXAMPLE V

### In Vivo Injection of Chitosan Sponges

A double-barrel, double-lumen syringe is used to administer the chitosan sponge in vivo. In brief, a 27-gauge needle is placed inside a 21-gauge needle and the head of the 21-gauge needle is trimmed until the tips of both needles are overlapping. The gap between the needle heads is sealed using epoxy. A metal tube is inserted between the two needle heads to provide outer flow of chitosan into the 21-gauge needle, while the 27-gauge needle provided the inner flow of GDP. Mixing took place at the tip of the needles during injection. The injection system is tested in a rat model with CSBD. A 6 mm segment of the femur is surgically removed and a fixator is attached to the two bone ends to prevent them from moving. The skin and soft tissue surrounding the cavity are then sutured back and the chitosan sponge is injected in the cavity. The sutures are then removed to observe the sponge and assess its localization.

While the invention has been described in connection with specific embodiments thereof, it will be understood that it is capable of further modifications and this application is intended to cover any variations, uses, or adaptations of the invention, including such departures from the present disclosure as come within known or customary practice within the art to which the invention pertains and as may be applied to the essential features hereinbefore set forth, and as follows in the scope of the appended claims.

What is claimed is:

1. A gelling composition comprising:
a) chitosan;
b) a purine compound; and
c) at least one of a growth factor and a pyrophosphatase.

2. The composition of claim 1, wherein the purine compound is at least one of a guanosine 5′-diphosphate (GDP), adenosine triphosphate (ATP), adenosine diphosphate (ADP), guanosine triphosphate (GTP), a substituted purine and a tautomer thereof.

3. The composition of claim 1, wherein the purine compound is guanosine 5′-diphosphate (GDP).

4. The composition of claim 1, wherein said composition comprises the growth factor.

5. The composition of claim 1, wherein said composition comprises the pyrophosphatase.

6. The composition of claim 1, wherein said composition comprises the growth factor and the pyrophosphatase.

7. The composition of claim 1, wherein the growth factor is at least one of a platelet-derived growth factor (PDGF), an insulin-like growth factor (IGF), a fibroblast growth factor (FGF), a transforming growth factor (TGF), an epidermal growth factor (EGF), a nerve growth factors (NGF), a vascular endothelial growth factor (VEGFs), and a bone morphogenetic protein (BMP).

8. The composition of claim 7, wherein the growth factor is at least one of PDGF, BMP-7, BMP-2, TGF-β, IGF-I, IGF-II, and bFGF.

11. The composition of claim 1, said composition being formulated for an injection.

12. A kit comprising:
a) a chitosan solution;
b) a solution containing at least one of a growth factor and a pyrophosphatase; and
b) a purine compound solution;
wherein a gel is formed when the chitosan solution and the solution containing at least one of the growth factor and the pyrophosphatase are mixed with the purine compound solution.

13. The kit of claim 12, wherein the chitosan solution, the solution containing at least one of the growth factor and the pyrophosphatase and the purine compound solution are manufactured in separate syringes; or wherein the chitosan solution and the solution containing at least one of the growth factor and the pyrophosphatase are manufactured in a double-barrel syringe.

14. The kit of claim 12, wherein the purine compound is at least one of a guanosine 5′-diphosphate (GDP), adenosine triphosphate (ATP), adenosine diphosphate (ADP), guanosine triphosphate (GTP), a substituted purine and a tautomer thereof.

15. The kit of claim 12, wherein the purine compound is guanosine 5′-diphosphate (GDP).

16. The kit of claim 12, wherein the growth factor is at least one of a platelet-derived growth factor (PDGF), an insulin-like growth factor (IGF), a fibroblast growth factor (FGF), a transforming growth factor (TGF), an epidermal growth factor (EGF), a nerve growth factors (NGF), a vascular endothelial growth factor (VEGFs), and a bone morphogenetic protein (BMP).

17. The kit of claim 12, wherein the growth factor is at least one of PDGF, BMP-7, BMP-2, TGF-β, IGF-I, IGF-II, and bFGF.

18. A method for stimulating bone regeneration in a subject comprising administering the composition of claim 1 or the kit of claim 12 to said subject.

19. The method of claim 18, wherein the subject has a fracture or a critical size bone defect (CSBD).

20. The method of claim 18, wherein the subject is an animal or a human.
